(19) United States
(12) Patent Application Publication
(10) Pub. No.: US 2024/0219054 A1
(43) Pub. Date: Jul. 4, 2024

(54) FAILURE DETERMINATION SYSTEM, FAILURE DETERMINATION METHOD, CONTROL DEVICE, AND PROGRAM

(71) Applicant: NIPPON TELEGRAPH AND TELEPHONE CORPORATION, Tokyo (JP)

(72) Inventors: Yasuhiro MATSUMOTO, Tokyo (JP); Satoshi SHINOZAKI, Tokyo (JP); Junichiro TAMAMATSU, Tokyo (JP)

(73) Assignee: NIPPON TELEGRAPH AND TELEPHONE CORPORATION, Tokyo (JP)

(21) Appl. No.: 18/558,108

(22) PCT Filed: May 13, 2021
(86) PCT No.: PCT/JP2021/018144

§ 371 Date: Oct. 30, 2023

(51) Int. Cl. F24F 11/49; F24F 110/76; G01N 33/00

(52) U.S. Cl. CPC F24F 11/49; G01N 33/0063; G01N 33/007; F24F 2110/76

(57) ABSTRACT

A failure determination system (100) according to the present disclosure includes a ventilation equipment (2); a gas concentration sensor (1) that outputs a detected value corresponding to a gas concentration, issues an alarm on the basis of the detected value, and transits alarm issuance information; and a control device (3). The control device (3) includes a communication unit (31) which receives alarm issuance information, a ventilation control unit (32) which controls the ventilation equipment (2) to execute a ventilation operation on the basis of the alarm issuance information, and a determination unit (33) which determines whether the gas concentration sensor (1) fails on the basis of the alarm issuance information.

### TECHNICAL FIELD

The present disclosure relates to a failure determination system, a failure determination method, a control device, and a program for determining a failure of a gas concentration sensor.

### BACKGROUND ART

In the related art, a gas sensor for sensing the concentration of a gas such as oxygen or a combustible gas is known (NPL 1). Also, a gas concentration sensor which is provided with such a gas sensor and issues an alarm when the concentration of the gas sensed by the gas sensor is not within a predetermined range is also known.

### CITATION LIST

### Non Patent Literature

[NPL 1] Tsuyoshi Ueda, “Principles, Structures and Features of Various Gas Sensors (Special Issue: Progress and New Applications of Gas Sensors),” Chemical Engineering of Japan, 81 (8), 410-413, 2017

### SUMMARY OF INVENTION

### Technical Problem

However, the gas concentration sensor may issue an alarm even if the gas concentration is within a predetermined range due to a failure due to deterioration or the like.

For example, in a gas concentration sensor which includes a gas sensor for sensing the concentration of oxygen and issues an alarm when the concentration of oxygen is less than a predetermined value, the sensitivity may decrease due to deterioration of the gas sensor, and a detected value lower than a normal detected value may be output. In this case, the gas concentration sensor issues an alarm even when the concentration of oxygen is equal to or higher than a predetermined value. In the gas concentration sensor that includes a gas sensor for sensing the concentration of the combustible gas and issues an alarm when the concentration of the combustible gas is a predetermined value or more, when a gas inlet to the gas sensor malfunctions, a larger amount of a gas may be detected than in a case in which the inlet is normal, thereby outputting a detected value higher than the normal detected value. In this case, the gas concentration sensor issues an alarm even when the concentration of the combustible gas is less than a predetermined value.

Therefore, it has been difficult to remotely determine whether an alarm has been issued because the gas concentration sensor was normal and the gas concentration was not within a predetermined range or whether an alarm has been issued due to failure of the gas concentration sensor.

An object of the present disclosure, which has been made in view of such circumstances, is to provide a failure determination system, a failure determination method, a control device, and a program that can remotely determine whether a gas concentration sensor has failed.

### Solution to Problem

In order to solve the above problem, a failure determination system according to the present disclosure includes ventilation equipment; a gas concentration sensor which outputs a detected value corresponding to a concentration of a gas in a space in which the ventilation equipment is provided, issues an alarm on the basis of the detected value, and transmits alarm issuance information regarding the issuance of the alarm; and a control device. The control device includes a communication unit which receives the alarm issuance information, a ventilation control unit which controls the ventilation equipment to execute a ventilation operation on the basis of the alarm issuance information, and a determination unit which determines whether the gas concentration sensor fails on the basis of the alarm issuance information after a ventilation completion time has elapsed from the start of the control.

In addition, in order to solve the above problem, a failure determination method according to the present disclosure includes a step of outputting a detected value corresponding to a concentration of a gas in a space in which ventilation equipment is provided; a step of issuing an alarm on the basis of the detected value; a step of controlling the ventilation equipment to execute a ventilation operation on the basis of the alarm issuance information; and a step of determining whether the gas concentration sensor fails on the basis of the alarm issuance information after a ventilation completion time has elapsed from the start of the control.

In order to solve the above problem, a control device according to the present disclosure includes a communication unit which outputs a detected value corresponding to a concentration of a gas in a space in which ventilation equipment is provided, and receives alarm issuance information related to issuance of an alarm transmitted from a gas concentration sensor that issues the alarm on the basis of the detected value; a ventilation control unit which controls the ventilation equipment to start a ventilation operation on the basis of the alarm issuance information, and a determination unit which determines whether the gas concentration sensor fails on the basis of the alarm issuance information after a ventilation completion time has elapsed from the control.

To solve the problems described above, a program according to the present disclosure causes a computer to function as the control device described above.

### Advantageous Effects of Invention

According to the failure determination system, the failure determination method, the control device, and the program according to the present disclosure, it is possible to remotely determine whether the gas concentration sensor fails.

### BRIEF DESCRIPTION OF DRAWINGS

FIG. 1 is a schematic diagram of a failure determination system according to a first embodiment of the present disclosure.

FIG. 2 is a diagram for explaining changes in detected values associated with failure of the gas concentration sensor shown in FIG. 1.

FIG. 3 is a sequence showing an example of an operation in the failure determination system shown in FIG. 1.

FIG. 4 is a flowchart for describing an example of execution of a sensing operation shown in FIG. 3 in detail.

FIG. 5 is a flowchart showing an example of failure determination shown in FIG. 3 in detail.

FIG. 6 is a schematic diagram of a failure determination system according to a second embodiment of the present disclosure.

FIG. 7 is a hardware block diagram of the control device.

### DESCRIPTION OF EMBODIMENTS

### First Embodiment

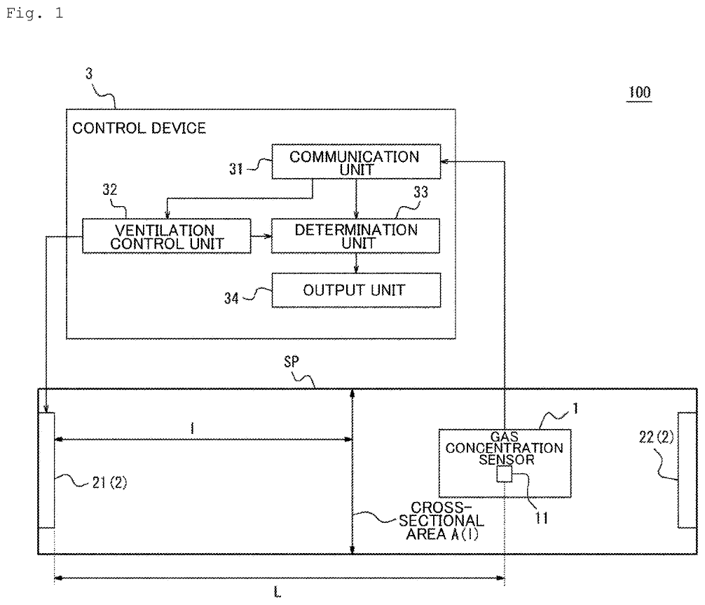

An overall configuration of a first embodiment will be described with reference to FIG. 1. FIG. 1 is a schematic diagram of a failure determination system 100 according to the present embodiment.

As shown in FIG. 1, the failure determination system 100 according to the first embodiment includes a gas concentration sensor 1, a ventilation equipment 2, and a control device 3. The control device 3 communicates with each of the gas concentration sensor 1 and the ventilation equipment 2 via a communication network.

### <Configuration of Gas Concentration Sensor>

The gas concentration sensor 1 is configured to include a gas sensor 11. The gas concentration sensor 1 is disposed in a space SP where the ventilation equipment 2 is provided. The space SP can be an arbitrary space defined by a structure, for example, it can be an underground tunnel. The gas concentration sensor 1 outputs a detected value corresponding to the concentration of the gas in the space SP where the ventilation equipment is provided, and issues an alarm on the basis of the detected value. Specifically, the gas concentration sensor 1 does not issue an alarm when the detected value is within a predetermined range, and issues an alarm when the detected value is outside of the predetermined range.

In an example, the gas whose concentration is detected by the gas concentration sensor 1 can be oxygen. In such a configuration, the predetermined range is equal to or greater than a first predetermined value. Therefore, the gas concentration sensor 1 does not issue an alarm when the detected value is equal to or higher than the first predetermined value, and issues an alarm when the detected value is less than the first predetermined value. Thus, a person staying in the space SP in which the gas concentration sensor 1 is disposed can recognize that the concentration of oxygen in the space SP is lower than the normal concentration of oxygen, and can take measures such as leaving the space SP or using a device for sucking oxygen.

In another example, the gas whose concentration is detected by the gas concentration sensor 1 can be a combustible gas. In such a configuration, the predetermined range is less than a second predetermined value. Therefore, the gas concentration sensor 1 issues an alarm when the detected value is equal to or higher than a second predetermined value, and the gas concentration sensor 1 issues no alarm when the detected value is less than the second predetermined value. Thus, a person staying in the space SP in which the gas concentration sensor 1 is disposed can recognize that the concentration of the combustible gas in the space SP is higher than the normal concentration of the combustible gas, and can take a countermeasure such as leaving the space SP or refraining from using firearms.

The alarm can be, for example, a sound. In a configuration in which the alarm is a sound, the sound may be an alarm sound or a sound indicating that a detected value is out of a predetermined range. The alarm may be, for example, an image displayed on a display device such as a liquid crystal or an organic EL. In the configuration in which the alarm is an image, the image may indicate that the detected value is outside of a predetermined range or may indicate information for urging the user to leave the space SP in which the gas concentration sensor 1 is disposed.

Further, the gas concentration sensor 1 transmits alarm issuance information related to the issuing of an alarm provided by the gas concentration sensor 1 to the control device 3.

In an example, the alarm issuance information may be alarm issuing information indicating that an alarm is being issued. In such a configuration, the gas concentration sensor 1 transmits alarm issuing information each time it detects that the concentration of the gas is not within a predetermined range. In another example, the alarm issuance information may be alarm start information and alarm stop information. In such a configuration, the gas concentration sensor 1 transmits alarm start information when issuance of the alarm is started, and transmits alarm issuance stop information when issuance of the alarm is stopped.

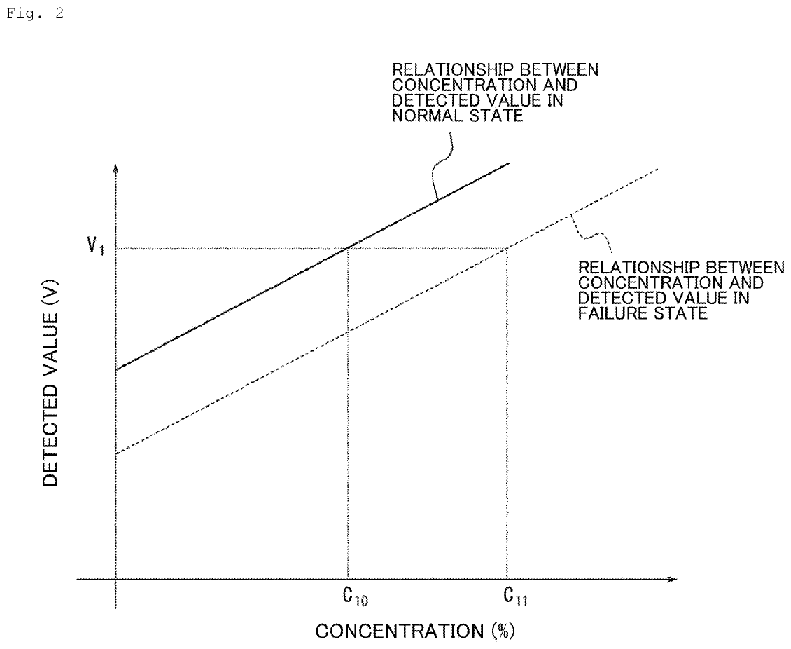

A relationship between the gas concentration and the value detected by the gas concentration sensor 1 will be described with reference to FIG. 2. The example shown in FIG. 2 shows the relationship between the gas concentration and the value detected by the gas concentration sensor 1 when the gas is oxygen. As shown in FIG. 2, the detected value becomes higher as the gas concentration becomes higher. Further, as shown by a broken line in FIG. 2, the value of the oxygen concentration detected by the gas concentration sensor 1 in the failure state is lower than the value of the oxygen concentration detected by the gas concentration sensor 1 in the normal state, as indicated by the solid line. Therefore, for example, in a case where it is necessary to alert a person staying in the space SP when the concentration of oxygen becomes less than C₁₀, the gas concentration sensor 1 is configured to issue an alarm when a detected value detected by the gas concentration sensor 1 is less than a first predetermined value V₁.

In such a configuration, in a case where the gas concentration sensor 1 is in a failure state, when the concentration of oxygen is less than Cu, which is higher than C₁₀, a detected value sensed by the gas concentration sensor 1 becomes less than a first predetermined value V₁. Therefore, the gas concentration sensor 1 issues an alarm when the concentration of oxygen is less than the predetermined value C₁₁. Thus, even when the concentration of oxygen is equal to or higher than a predetermined value C₁₀ and safe, a person staying in the space SP may be alerted unnecessarily.

Similarly, when the gas is a combustible gas, a value of the concentration of the combustible gas detected by the gas concentration sensor 1 in the failure state is higher than the value of the combustible gas concentration detected by the gas concentration sensor 1 in the normal state. Therefore, for example, in a case where it is necessary to alert a person staying in the space SP when the concentration of the combustible gas becomes C₂₀ or more, the gas concentration sensor 1 is configured to issue an alarm when a detected value detected by the gas concentration sensor 1 is a second predetermined value V₂ or more.

In such a configuration, in a case where the gas concentration sensor 1 is in the failure state, when the concentration of the combustible gas is equal to or higher than C₂₁, which is lower than C₂₀, a detected value sensed by the gas concentration sensor 1 becomes a second predetermined value V₂ or more. Therefore, the gas concentration sensor 1 issues an alarm when the concentration of the combustible gas is C₂₁ or more. Thus, even when the concentration of the combustible gas is less than the predetermined value C₂₀ and safe, a person staying in the space SP may be alerted unnecessarily.

Hereinafter, the ventilation equipment 2 and the control device 3 used for determining whether the gas concentration sensor 1 fails will be described in detail.

### <Configuration of Ventilation Equipment>

The ventilation equipment 2 is disposed in the space SP. For example, the ventilation equipment 2 may be attached to a member such as a wall, a ceiling or the like that partitions the space SP from the outside. The ventilation equipment 2 includes inflow side ventilation equipment 21 and outflow side ventilation equipment 22.

The inflow side ventilation equipment 21 is ventilation equipment for making a gas flow in from the outside, for example, a ventilation fan.

The outflow side ventilation equipment 22 is ventilation equipment for making a gas in the space SP flow out to the outside, and may be a ventilation fan or a ventilation port.

### <Configuration of Control Device>

The control device 3 includes a communication unit 31, a ventilation control unit 32, a determination unit 33, and an output unit 34. The communication unit 31 is constituted by a communication interface for communicating with another apparatus such as an external device. For the communication interface, standards such as Ethernet (registered trademark), Fiber Distributed Data Interface (FDDI), Wi-Fi (registered trademark), and the like may be used. The ventilation control unit 32 and the determination unit 33 constitute a control unit (controller). The control unit may be constituted by dedicated hardware such as an application specific integrated circuit (ASIC) and a field-programmable gate array (FPGA), may be constituted by a processor, or may include both. The output unit 34 may be constituted by a communication interface, a voice output interface, or a display interface. The voice output interface may be a speaker or the like. The display interface is an interface for displaying information on a display device made up of a liquid crystal panel, an organic EL or the like.

The communication unit 31 receives the alarm issuance information. As described above, in the configuration in which the alarm issuance information is the alarm issuing information, the communication unit 31 receives the alarm issuing information transmitted when the gas concentration sensor 1 issues an alarm. In a configuration in which the alarm issuance information is the alarm start information and the alarm stop information, the communication unit 31 receives the alarm start information transmitted when the gas concentration sensor 1 starts issuing an alarm, and receives the alarm stop information transmitted when the gas concentration sensor 1 stops issuing the alarm.

The ventilation control unit 32 controls the ventilation equipment 2 to execute a ventilation operation on the basis of the alarm issuance information received by the communication unit 31.

Specifically, the ventilation control unit 32 controls the ventilation equipment 2 to execute the ventilation operation when the alarm issuance information indicates that the alarm is issued by the gas concentration sensor 1. For example, the ventilation control unit 32 performs control so that the ventilation equipment 2 performs the ventilation operation when the alarm issuance information is alarm issuing information. Further, for example, when the alarm issuance information received by the communication unit 31 is alarm issuance start information and the alarm stop information is not received after the reception of the alarm issuance start information, the ventilation control unit 32 performs a control so that the ventilation equipment 2 performs the ventilation operation.

The ventilation control unit 32 controls the ventilation equipment 2 not to execute the ventilation operation when the alarm issuance information indicates that the alarm is not issued by the gas concentration sensor 1. For example, the ventilation control unit 32 performs a control so that the ventilation equipment 2 does not execute the ventilation operation when the alarm issuing information is not received. Further, for example, when the alarm issuance information received by the communication unit 31 is the alarm issuance stop information and the alarm start information is not received after the reception of the alarm issuance information, the ventilation control unit 32 controls the ventilation equipment 2 such that the ventilation operation is not executed.

More specifically, the ventilation control unit 32 turns a ventilation fan which is the inflow side ventilation equipment 21 in the control for making the ventilation equipment 2 execute a ventilation operation. In a configuration in which the outflow side ventilation equipment 22 is a ventilation fan, the ventilation control unit 32 may turn the outflow side ventilation equipment 22 in the control for making the ventilation equipment 2 execute a ventilation operation. In the configuration in which the outflow side ventilation equipment 22 is the ventilation port, the ventilation control unit 32 may open the outflow side ventilation equipment 22 in the control for making the ventilation equipment 2 execute the ventilation operation. In FIG. 1, arrows from the ventilation control unit 32 to the outflow side ventilation equipment 22 are omitted.

The determination unit 33 determines whether the gas concentration sensor 1 fails on the basis of the alarm issuance information after a ventilation completion time t1 has elapsed from the start of control for making the ventilation equipment 2 execute the ventilation operation. The ventilation completion time t1 is a time required for exchanging gas whose concentration is detected by the gas concentration sensor 1 with external gas. The ventilation completion time t1 is a value obtained by dividing a volume from the inflow side ventilation equipment 21 to the gas sensor 11 of the gas concentration sensor 1 in the space SP by an air volume Q(m/s) of the gas flowing in from the external environment per unit time, as shown in the following Formula (1). The volume from the inflow side ventilation equipment 21 to the gas sensor 11 of the gas concentration sensor 1 is a value obtained by integrating a cross-sectional area A(l) of the space SP orthogonal to the line segment from the inflow side ventilation equipment 21 to the gas sensor 11 of the gas concentration sensor 1 by a distance l from the inflow side ventilation equipment 21, as shown in FIG. 1. In the Formula (1), L represents a distance from the inflow side ventilation equipment 21 to the gas sensor 11 of the gas concentration sensor 1.

$$\begin{matrix}[ Math . 1 ] & \\ t 1 = \frac{{\int }_{0}^{L} A ( 1 ) dl}{Q} & ( 1 )\end{matrix}$$

When the cross-sectional area A(l) of the space SP is a constant value A independent of the distance l, the ventilation completion time t1 is expressed by the following Formula (2).

$$\begin{matrix}[ Math . 2 ] & \\ t 1 = \frac{AL}{Q} & ( 2 )\end{matrix}$$

The determination unit 33 determines that the gas concentration sensor 1 fails when it is determined that issuance of the alarm is continued after the ventilation completion time t1 has elapsed. The determination unit 33 determines that the gas concentration sensor 1 does not fail when it is determined that issuance of the alarm is not continued after the ventilation completion time t1 has elapsed.

In an example, the determination unit 33 determines whether the communication unit 31 continuously receives the alarm issuing information after the lapse of a ventilation completion time t1 after the ventilation equipment 2 starts a control to execute the ventilation operation. When the communication unit 31 continuously receives the alarm issuing information, the determination unit 33 determines that the gas concentration sensor 1 fails. The determination unit 33 determines that the gas concentration sensor 1 does not fail when the communication unit 31 does not receive the alarm issuing information.

In another example, the determination unit 33 determines whether the communication unit 31 has received the alarm stop information from the start of control so that the ventilation equipment 2 executes the ventilation operation to the lapse of the ventilation completion time t1. The determination unit 33 determines that the gas concentration sensor 1 fails, when the communication unit 31 does not receive the alarm stop information from the start of control for the ventilation equipment 2 to execute the ventilation operation to the lapse of the ventilation completion time t1. The determination unit 33 determines that the gas concentration sensor 1 does not fail, when the communication unit 31 receives the alarm stop information from the start of the control of the ventilation equipment 2 to execute the ventilation operation to the lapse of the ventilation completion time t1.

The output unit 34 outputs a result determined by the determination unit 33. Specifically, the output unit 34 can display the result determined by the determination unit 33. The output unit 34 may output a sound indicating the result determined by the determination unit 33. The output unit 34 may transmit the result determined by the determination unit 33 to another device via a communication network.

### <Operation of Failure Determination System>

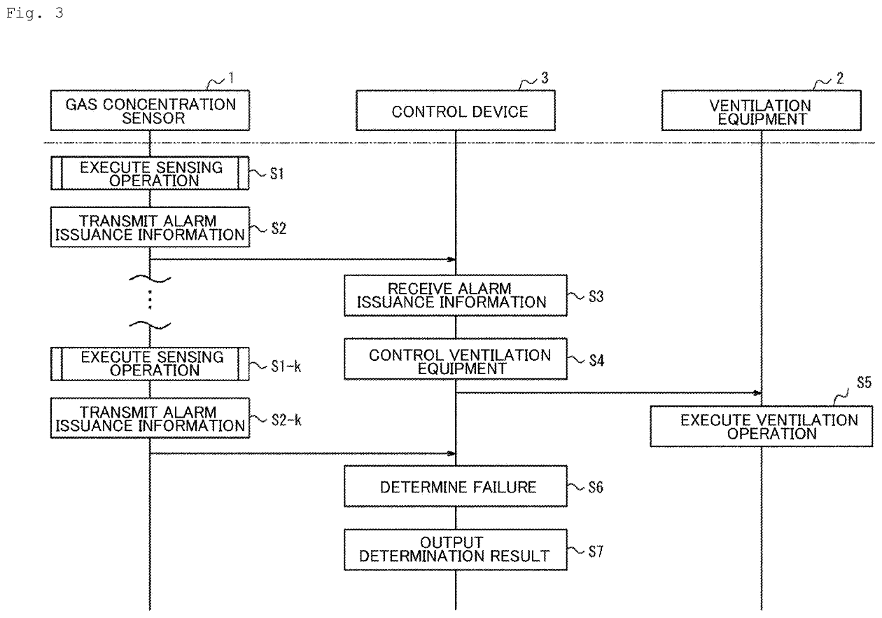

Here, an operation of the failure determination system 100 according to the first embodiment will be described referring to FIG. 3. FIG. 3 is a sequence diagram showing an example of the operation in the failure determination system 100 according to the first embodiment. The operation in the failure determination system 100 described with reference to FIG. 3 corresponds to the failure determination method of the failure determination system 100 according to the first embodiment.

In step S1, the gas concentration sensor 1 executes a sensing operation.

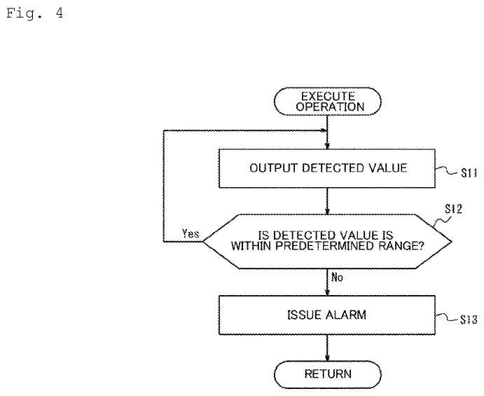

The sensing operation executed by the gas concentration sensor 1 will now be described in detail with reference to FIG. 4.

In step S11, the gas concentration sensor 1 outputs a detected value corresponding to the concentration of the gas in the space SP provided with the ventilation equipment 2.

In steps S12 and S13, the gas concentration sensor 1 issues an alarm on the basis of the detected value.

Specifically, in step S12, first, the gas concentration sensor 1 determines whether the detected value is within a predetermined range.

When it is determined in step S12 that the detected value is within the predetermined range, the gas concentration sensor 1 returns to the step S11 and repeats the operation. When it is determined that the detected value is out of the predetermined range in the step S12, the gas concentration sensor 1 issues an alarm in step S13.

Returning to FIG. 3, in step S2, the gas concentration sensor 1 transmits the alarm issuance information to the control device 3.

Thereafter, the gas concentration sensor 1 executes a sensing operation at a predetermined timing (step S1-k (k is an integer from 1 to N)), and when an alarm is issued in the sensing operation, the gas concentration sensor 1 repeats transmission of the alarm issuance information (step S2-k).

In step S3, the communication unit 31 of the control device 3 receives the alarm issuance information.

In step S4, the ventilation control unit 32 of the control device 3 performs a control so that the ventilation equipment 2 executes the ventilation operation on the basis of the alarm issuance information.

In step S5, the ventilation equipment 2 executes the ventilation operation.

In step S6, the determination unit 33 of the control device 3 determines whether the gas concentration sensor 1 fails on the basis of the alarm issuance information after the ventilation completion time t1 has elapsed from the start of control.

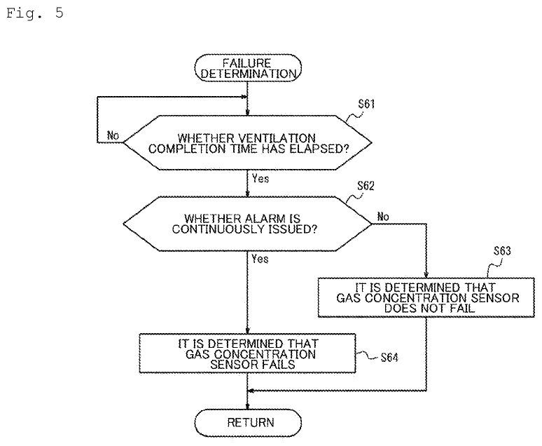

Next, the failure determination executed by the determination unit 33 of the control device 3 will be described in detail with reference to the flowchart of FIG. 5.

In step S61, the determination unit 33 determines whether the ventilation completion time t1 has elapsed since the control for causing the ventilation equipment 2 to execute the ventilation operation.

When it is determined in the step S61 that the ventilation completion time t1 has not elapsed after start of the control for making the ventilation equipment 2 execute the ventilation operation, the determination unit 33 executes the processing of the step S61 again. When it is determined in the step S61 that the ventilation completion time t1 has elapsed after the start of control for making the ventilation equipment 2 execute the ventilation operation, in step 62, the determination unit 33 determines whether the alarm is continuously issued.

When it is determined in the step S62 that the alarm is not continuously issued, the determination unit 33 determines that the gas concentration sensor 1 does not fail in the step S63.

When it is determined that the alarm is continuously issued in the step S62, the determination unit 33 determines that the gas concentration sensor 1 fails in step S64.

Returning to FIG. 3, in step S7, the output unit 34 outputs a determination result indicating whether the gas concentration sensor 1 fails.

As described above, according to the first embodiment, the failure determination system 100 receives the alarm issuance information and controls the ventilation equipment 2 based on the alarm issuance information. Then, the failure determination system 100 determines whether the gas concentration sensor 1 fails on the basis of the alarm issuance information after the ventilation completion time t1 has elapsed from the start of control for making the ventilation equipment 2 execute the ventilation operation. Therefore, the failure determination system 100 can determine whether the gas concentration sensor 1 fails remotely.

### Second Embodiment

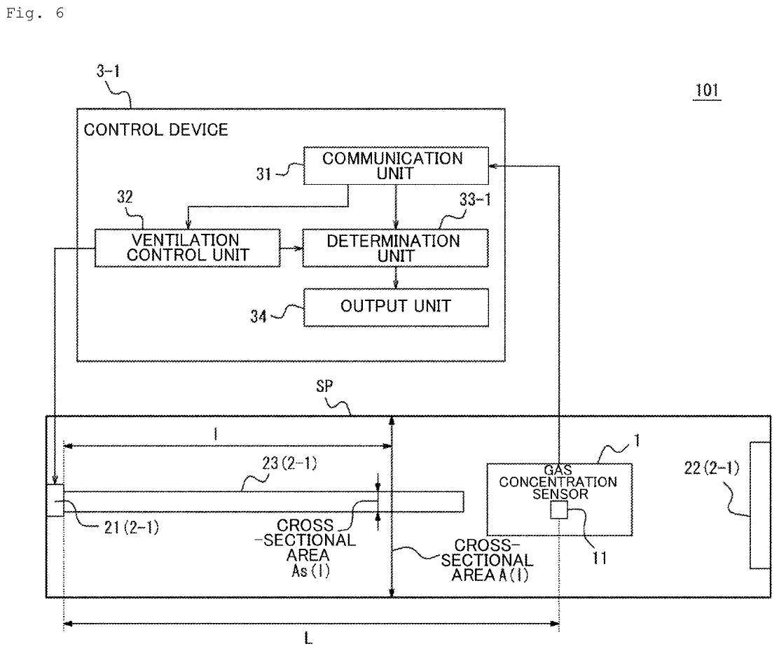

An overall configuration of a second embodiment will be described with reference to FIG. 6. FIG. 6 is a schematic diagram of a failure determination system 101 according to the second embodiment. The same components of the second embodiments as those of the first embodiment are denoted by the same reference numerals and description thereof will not be provided.

As shown in FIG. 6, the failure determination system 101 according to the second embodiment includes a gas concentration sensor 1, a ventilation equipment 2-1, and a control device 3-1. The control device 3-1 communicates with the gas concentration sensor 1 and the ventilation equipment 2-1 via a communication network.

### <Configuration of Ventilation Equipment>

The ventilation equipment 2-1 is disposed in the space SP as in the ventilation equipment 2 of the first embodiment. The ventilation equipment 2-1 includes an inflow side ventilation equipment 21, an outflow side ventilation equipment 22, and a pipe 23.

The pipe 23 is disposed in the space SP and allows gas flowing in from the outside of the space SP to pass toward the gas concentration sensor 1. In an example, as shown in FIG. 6, the pipe 23 extends from the inflow side ventilation equipment 21 to the gas concentration sensor 1. In such an example, a cross-sectional area As(l) of the pipe 23 is smaller than the cross-sectional area A(l) of the space SP at an arbitrary distance l in a direction from the inflow side ventilation equipment 21 toward the gas concentration sensor 1. In the example shown in FIG. 6, the cross-sectional area As(l) of the pipe 23 is a constant value As independent of the distance l.

### <Configuration of Control Device>

The control device 3-1 includes a communication unit 31, a ventilation control unit 32, a determination unit 33-1 and an output unit 34. The determination unit 33-1 constitutes a control unit (controller) in the same manner as the determination unit 33 of the first embodiment.

The determination unit 33-1 determines whether the gas concentration sensor 1 fails on the basis of the alarm issuance information after a ventilation completion time t2 has elapsed after a control for making the ventilation equipment 2 execute the operation is started. The ventilation completion time t2 is a time required for exchanging gas whose concentration is detected by the gas concentration sensor 1 with external gas. The ventilation completion time t2 is expressed by the following Formula (3). L in Formula (3) is a distance from the inflow side ventilation equipment 21 to the gas sensor 11 of the gas concentration sensor 1, and As is a cross-sectional area of the pipe 23. Qs is an air volume (m/s) of the gas flowing into the pipe 23 per unit time from the external environment.

$$\begin{matrix}[ Math . 3 ] & \\ t 2 = \frac{AsL}{Qs} & ( 3 )\end{matrix}$$

Formula (3) indicates the ventilation completion time t2 in an example in which the cross-sectional area of the pipe 23 is constant. When the cross-sectional area As of the pipe 23 is not constant but is As(l) depending on the distance l, the ventilation completion time t2 can be a value obtained by dividing the value, which is obtained by integrating the cross-sectional area As(l) of the pipe 23 by the distance l from the inflow side ventilation equipment 21, by the air volume Qs (m/s).

The shape and placement of the pipe 23 are not limited to the example shown in FIG. 6, and one end of the pipe 23 may be located in the ventilation equipment 2 and the other end thereof may be located at an arbitrary point between the ventilation equipment 2 and the gas concentration sensor 1. For example, the pipe 23 may be disposed such that one end of the pipe 23 is located near the ventilation equipment 2 and the other end thereof is located at an intermediate point between the ventilation equipment 2 and the gas concentration sensor 1.

The determination unit 33-1 determines that the gas concentration sensor 1 fails when it is determined that issuance of the alarm is continued after the ventilation completion time t2 has elapsed. The determination unit 33-1 determines that the gas concentration sensor 1 does not fail when it is determined that issuance of the alarm is not continued after the ventilation completion time t2 has elapsed. The details of the determination method performed by the determination unit 33-1 are the same as the details of the determination method performed by the determination unit 33 except the ventilation completion time t2.

### <Operation of Failure Determination System>

The operation of the failure determination system 101 according to the second embodiment is the same as the operation of the failure determination system 100 according to the first embodiment except that the ventilation completion time t2 is used instead of the ventilation completion time t1.

As described above, according to the second embodiment, the failure determination system 101 includes the pipe 23 that is disposed in the space SP and allows the gas introduced from the outside of the space SP to pass toward the gas concentration sensor 1. Therefore, the gas flowing in from the outside of the space SP can efficiently reach the gas concentration sensor 1, and therefore, the gas detected by the gas concentration sensor 1 is efficiently exchanged with the gas flowing in from the outside of the space SP. Therefore, the failure determination system 101 can determine whether the gas concentration sensor 1 has failed in a shorter time than the failure determination system 100 of the first embodiment.

### Program

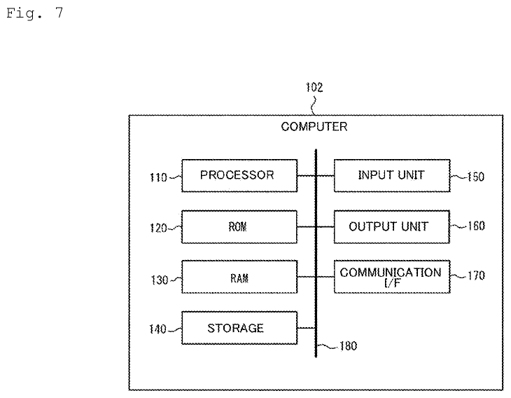

The control devices 3 and 3-1 described above can be realized by the computer 102. Further, a program for functioning as each of the control devices 3 and 3-1 may be provided. The program may be stored on a storage medium or provided through a network. FIG. 7 is a block diagram showing a schematic configuration of a computer 102 which functions as each of the control devices 3 and 3-1. Here, the computer 102 may be a general-purpose computer, a dedicated computer, a work station, a personal computer (PC), an electronic note pad, or the like. The program instructions may be program codes, code segments, or the like for executing necessary tasks.

As shown in FIG. 7, the computer 102 includes a processor 110, a read only memory (ROM) 120, a random access memory (RAM) 130, a storage 140, an input unit 150, an output unit 160, and a communication interface (I/F) 170. Each configuration is connected to each other via a bus 180 to be communicable with each other. Specifically, the processor 110 may be a central processing unit (CPU), a micro processing unit (MPU), a graphics processing unit (GPU), a digital signal processor (DSP), a System on a Chip (SoC), and the like, and may be constituted by a plurality of processors of the same kind or different kinds.

The processor 110 executes control of each configuration and various calculation processing. That is, the processor 110 reads a program from the ROM 120 or the storage 140 and executes the program using the RAM 130 as a working area. The processor 110 performs control of each of the above components and various calculation processing in accordance with programs stored in the ROM 120 or the storage 140. In the embodiment, the ROM 120 or the storage 140 stores a program according to the present disclosure.

The program may also be recorded on a recording medium which can be read by the computer 102. When using such a recording medium, it is possible to install the program on the computer 102. Here, the recording medium on which the program is recorded may be a non-transitory recording medium. Although not particularly limited, the non-transitory recording medium may be, for example, a CD-ROM, a DVD-ROM, a universal serial bus (USB) memory, or the like. Also, this program may be downloaded from an external device over a network.

The ROM 120 stores various programs and various types of data. The RAM 130 is a work area and temporarily stores a program or data. The storage 140 is configured by a hard disk drive (HDD) or a solid state drive (SSD), and stores various programs including an operating system and various types of data.

The input unit 150 includes one or more input interfaces through which user's input operations are received and information based on the user's operations is acquired. For example, the input unit 150 is a pointing device, a keyboard, a mouse, and the like, but is not limited to these.

The output unit 160 includes one or more output interfaces through which information is output. For example, the output unit 160 is a display for outputting information as a video or a speaker for outputting information as a sound, but is not limited thereto. The output unit 160 also serves as the input unit 150 in the case of a touch panel type display.

A communication interface 170 is an interface for communicating with an external device.

The following appendices are disclosed in relation to the embodiments described above.

### Appendix 1

A failure determination system including:
a ventilation equipment;
a gas concentration sensor which outputs a detected value corresponding to a concentration of gas in a space in which the ventilation equipment is provided, issues an alarm on the basis of the detected value, and transits alarm issuance information related to the issuance of the alarm; and
a control device,
wherein the control device receives the alarm issuance information
the control device controls the ventilation equipment to execute a ventilation operation on the basis of the alarm issuance information, and
the control device determines whether the gas concentration sensor fails on the basis of the alarm issuance information after a ventilation completion time has elapsed from the start of the control.

### Appendix 2

The failure determination system described in Appendix 1, wherein the control device determines that the gas concentration sensor fails when it is determined that issuance of the alarm is continued after the ventilation completion time has elapsed, and the control device determines that the gas concentration sensor does not fail when it is determined that issuance of the alarm is not continued after the ventilation completion time has elapsed.

### Appendix 3

The failure determination system described in any one of Appendices 1 to 3, further including a pipe which is disposed in the space and causes the gas introduced from the outside of the space to pass toward the gas concentration sensor.

### Appendix 4

A failure determination method including:
a step of outputting a detected value corresponding to a concentration of gas in a space in which a ventilation equipment is provided;
a step of issuing an alarm on the basis of the detected value;
a step of controlling the ventilation equipment to execute a ventilation operation on the basis of alarm issuance information related to an issuance of the alarm; and
a step of determining whether the gas concentration sensor fails on the basis of the alarm issuance information after a ventilation completion time has elapsed from the start of the control.

### Appendix 5

A control device including:
a communication unit which outputs a detected value corresponding to a concentration of gas in a space in which a ventilation equipment is provided, and receives alarm issuance information related to issuance of an alarm transmitted from a gas concentration sensor that issues the alarm on the basis of the detected value; and
a control unit,
wherein the control unit
controls the ventilation equipment to start a ventilation operation on the basis of the alarm issuance information, and determines whether the gas concentration sensor fails on the basis of the alarm issuance information after a ventilation completion time has elapsed from the control.

### Appendix 6

A non-transitory storage medium which stores a program executable by a computer, and the program causes the computer to function as the control device described in Appendix 5.

All documents, patent applications, and technical standards mentioned in this specification are incorporated herein by reference to the same extent as if each individual document, patent application, or technical standard were specifically and individually indicated to be incorporated by reference.

Although the above-described embodiment has been introduced as a typical example, it is clear for a person skilled in the art that many alterations and substitutions are possible within the gist and scope of the present disclosure. Therefore, the embodiment described above should not be interpreted as limiting, and the present invention can be modified and altered in various ways without departing from the scope of the claims. For example, a plurality of configuration blocks shown in the configuration diagrams of the embodiments may be combined to one, or one configuration block may be divided.

### REFERENCE SIGNS LIST

1 Gas concentration sensor
11 Gas sensor
2, 2-1 Ventilation equipment
3, 3-1 Control device
21 Inflow side ventilation equipment
22 Outflow side ventilation equipment
23 Pipe
31 Communication unit
32 Ventilation control unit
33, 33-1 Determination unit
34 Output unit
100, 101 Congestion information processing system
102 Computer
110 Processor
120 ROM
130 RAM
140 Storage
150 Input unit
160 Output unit
170 Communication interface
180 Bus

1. A failure determination system comprising:
ventilation equipment;
a gas concentration sensor comprising a first processor configured to execute operations comprising:
outputting a detected value corresponding to a concentration of a gas in a space in which the ventilation equipment is provided,
generating an alarm on the basis of the detected value, and
transmitting alarm issuance information regarding the issuance of the alarm; and
a control device comprising a second processor configured to execute operations comprising:
receiving the alarm issuance information,
causing the ventilation equipment to execute a ventilation operation on the basis of the alarm issuance information, and
determining whether the gas concentration sensor fails on the basis of the alarm issuance information after a ventilation completion time has elapsed from the start of the causing the ventilation equipment to execute the ventilation operation.

2. The failure determination system according to claim 1,
wherein the determining further comprises determining a failure of the gas concentration sensor when it is determined that issuance of the alarm continues after the ventilation completion time has elapsed, and
the determining further comprises determining a non-failure of the gas concentration sensor when it is determined that issuance of the alarm does not continue after the ventilation completion time has elapsed.

3. The failure determination system according to claim 1, further comprising:
a pipe which is disposed in the space and causes the gas introduced from the outside of the space to pass toward the gas concentration sensor.

4. A failure determination method comprising:
a step of outputting a detected value corresponding to a concentration of a gas in a space in which ventilation equipment is provided;
a step of issuing an alarm on the basis of the detected value;
a step of causing the ventilation equipment to execute a ventilation operation on the basis of alarm issuance information related to issuance of the alarm; and
a step of determining whether a gas concentration sensor fails on the basis of the alarm issuance information after a ventilation completion time has elapsed from the start of the causing the ventilation equipment to execute the ventilation operation.

5. A control device comprising a processor configured to execute operations comprising:
outputting a detected value corresponding to a concentration of a gas in a space in which ventilation equipment is provided;
receiving alarm issuance information related to issuance of an alarm transmitted from a gas concentration sensor that issues the alarm on the basis of the detected value;
causing the ventilation equipment to start a ventilation operation on the basis of the alarm issuance information; and
determining whether the gas concentration sensor fails on the basis of the alarm issuance information after a ventilation completion time has elapsed from the causing the ventilation equipment to execute the ventilation operation.

6. (canceled)

7. The failure determination system according to claim 1, wherein the gas represents oxygen, and the issuance of the alarm from the gas concentration sensor is according to the detected value being less than a threshold value.

8. The failure determination system according to claim 1, wherein the gas represents a combustible gas, and the issuance of the alarm from the gas concentration sensor is according to the detected value being greater than a threshold value.

9. The failure determination system according to claim 2, further comprising:
a pipe which is disposed in the space and causes the gas introduced from the outside of the space to pass toward the gas concentration sensor.

10. The failure determination system according to claim 2, wherein the failure of the gas concentration sensor represents deterioration of the gas concentration sensor.

11. The failure determination method according to claim 4, wherein the gas represents oxygen, and the issuance of the alarm from the gas concentration sensor is according to the detected value being less than a threshold value.

12. The failure determination method according to claim 4, wherein the gas represents a combustible gas, and the issuance of the alarm from the gas concentration sensor is according to the detected value being greater than a threshold value.

13. The failure determination method according to claim 4, wherein the step of determining further comprises determining a failure of the gas concentration sensor when it is determined that issuance of the alarm continues after the ventilation completion time has elapsed, and
the step of determining further comprises determining a non-failure of the gas concentration sensor when it is determined that issuance of the alarm does not continue after the ventilation completion time has elapsed.

14. The failure determination method according to claim 13, wherein the failure of the gas concentration sensor represents deterioration of the gas concentration sensor.

15. The control device according to claim 5, wherein the gas represents oxygen, and the issuance of the alarm from the gas concentration sensor is according to the detected value being less than a threshold value.

16. The control device according to claim 5, wherein the gas represents a combustible gas, and the issuance of the alarm from the gas concentration sensor is according to the detected value being greater than a threshold value.

17. The control device according to claim 5, wherein the step of determining further comprises determining a failure of the gas concentration sensor when it is determined that issuance of the alarm continues after the ventilation completion time has elapsed, and
the step of determining further comprises determining a non-failure of the gas concentration sensor when it is determined that issuance of the alarm does not continue after the ventilation completion time has elapsed.

18. The control device according to claim 17, wherein the failure of the gas concentration sensor represents deterioration of the gas concentration sensor.
